(19) United States
(12) Patent Application Publication
(10) Pub. No.: US 2010/0106216 A1
(43) Pub. Date: Apr. 29, 2010

(54) PROBE FOR ELECTRIC STIMULATOR HAVING VARIABLE VAGINAL ELECTRODE

(76) Inventors: Eun Jong Cha, Cheongju-City (KR); Seung Jik Lee, Jecheon-City (KR); Kyung Ah Kim, Cheongju-City (KR)

Correspondence Address: GREER, BURNS & CRAIN, 300 S WACKER DR, 25TH FLOOR, CHICAGO, IL, 60606, US

(21) Appl. No.: 12/485,454

(22) Filed: Jun. 16, 2009

(30) Foreign Application Priority Data
Oct. 29, 2008 (KR) 10-2008-0106217

(51) Int. Cl. A61N 1/04

(52) U.S. Cl. 607/41

(57) ABSTRACT

A probe for an electric stimulator having a variable vaginal electrode constantly maintains the contact resistance by adjusting the diameter of the electrode corresponding to the user. Electrodes are provided on front and rear parts of a body of the probe, respectively. The electrodes are provided in a circular arrangement such that the electrodes move up and down. A rotating handle is formed at a side of the body such that the handle rotates at the side of the body. A shaft coupled to the handle rotates when the handle rotates, so a cam plate coupled to the shaft rotates, and the rotated cam plate pushes the electrodes, thereby expanding or reducing a diameter of the electrodes.

### BACKGROUND OF THE INVENTION

1. Field of the Invention

The present invention relates to a probe for an electric stimulator having a variable vaginal electrode, capable of enhancing a treatment efficiency when treating patients suffering from urinary incontinence using an electric stimulator by changing a diameter of an electrode corresponding to the patients in order to prevent the treatment efficiency from being lowered due to a change of a contact resistance between the electrode and a coelome of the patients.

2. Description of the Prior Art

In general, an electrical stimulation, which is performed to apply electrical stimulus to a pelvic floor and an adjacent tissue by inserting an electrode into a vagina of a woman, is known as an effective treatment for urinary incontinence, such as stress incontinence and urge incontinence.

The medical electrotherapy uses galvanic electricity, induced electricity and alternative electricity, and has various functions and purposes according to waveform of a frequency, current intensity and affected parts.

Recently, a high frequency electric stimulator and a low frequency electric stimulator, which are classified according to frequency thereof, have been extensively developed and used in various fields. A low frequency electrical treatment uses a frequency within a range of several Hz to 1000 Hz, and a high frequency electrical treatment uses a frequency of 10,000 Hz or above.

For example, if the high frequency current is applied to a human body, deep heat is generated from a skin tissue of the human body. This is because that if electric energy having high frequency is applied, whenever a direction of current is changed, molecules constituting the tissue make friction with each other while vibrating, thereby causing rotation, twist and collision, so that in-vivo heat is generated.

In the electric stimulator, an electrode is necessary for applying electricity to the human body. FIGS. 1 and 2 show a shape and a structure of a probe having the electrode inserted into a vagina.

As shown in FIGS. 1 and 2, a conventional probe for an electric stimulator includes a body 1, which is molded by using plastic and is provided at a surface thereof with two circular electrodes 2a and 2b having a width of about 1 cm.

In the interior of the probe, two lines 3a and 3b are simply connected to an external terminal and the two lines 3a and 3b are connected to the electrodes 2a and 2b, respectively.

Such a probe is inserted into a coelome of a patient. Since a diameter of the probe is fixed to about 25 mm, when the patient moves in a state that the probe is inserted into the coelome, the probe may slip out of the coelome.

In addition, a size of the coelome varies depending on the patient, so a contact resistance between a skin and the electrode may vary depending on the patient.

If the contact resistance varies, the transmission efficiency of stimulus is lowered. For this reason, the contact resistance becomes an important factor to appropriately transfer the pulses of the electrical stimulus to muscles inside the coelome.

That is, in order to treat all urinary incontinence patients under the same stimulus condition, the contact resistance must be constantly maintained for all patients. However, according to the conventional electric stimulator, a diameter of the electrode probe is fixed, so it is difficult to constantly maintain the contact resistance for all patients.

### SUMMARY OF THE INVENTION

Accordingly, it is an aspect of the present invention to provide a probe for an electric stimulator having a variable vaginal electrode, capable of constantly maintaining the contact resistance by adjusting a diameter of an electrode corresponding to use environment.

Additional aspects and/or advantages of the disclosure will be set forth in part in the description, which follows and, in part, will be apparent from the description, or may be learned by practice of the disclosure.

The foregoing and/or other aspects of the disclosure are achieved by providing a probe for an electric stimulator having a variable vaginal electrode. The probe includes a body divided into a front part and a rear part, and electrodes provided on the front and rear parts, respectively. A plurality of electrodes are provided in a circular arrangement such that the electrodes move up and down, a rotating handle is formed at a side of the body such that the handle rotates at the side of the body, a shaft coupled to the handle rotates when the handle rotates, so a cam plate coupled to the shaft rotates, and the rotated cam plate pushes the electrodes, thereby expanding or reducing a diameter of the electrodes.

A return spring 150 is installed between the electrodes 100a and 100b and the body 110.

The shaft 130 includes a first shaft 130a, which is fixed to the handle 120 to extend toward a front end of the rear part 114, and a second shaft, which is connected to the first shaft 130a through a joint 132 to extend to an inside of the front part 112, and two cam plates 140 having an S shape are coupled to the first shaft 130a and the second shaft 130b, respectively, while crossing each other such that a lower end of the electrodes is mounted on the two cam plates 140.

A power cable introduced through the handle 120 has a first pole, which makes contact with the first shaft 130a and communicates with the electrode 100b of the rear part 114, and a second pole, which passes through an inside of the first shaft 130a and makes contact with the second shaft 130b through a contact terminal 135 formed in the joint 132 so that the second pole communicates with the electrode 100a of the front part 112.

A sliding protrusion 102 having a semicircular shape is formed at a lower surface of the electrodes 100a and 100b.

The joint 132 includes insulating material, the contact terminal 134 is elastically supported by a compression spring at a front end of the first shaft 130a, and a tip of the contact terminal 135 makes contact with the second shaft 130b.

According to a probe for an electric stimulator having a variable vaginal electrode of the present invention, the rotation movement of a handle is transferred to a shaft, and the rotation movement of the shaft causes expansion or reduction in a diameter of an electrode, so that the diameter of the electrode can be adjusted corresponding to user environment. Accordingly, the electric stimulator can constantly maintain the contact resistance.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a perspective view showing a probe for an electric stimulator according to the related art;

FIG. 2 is a sectional view showing an interior of the probe shown in FIG. 1;

FIG. 3 is a schematic view showing a probe for an electric stimulator according to the present invention;

FIG. 4 is a view showing an interior of the probe for the electric stimulator according to the present invention;

FIG. 5 is a view showing a cam plate shown in FIG. 4;

FIG. 6 is a view showing an electrode which moves up and down according to a rotation of the cam plate; and

FIG. 7 is a view showing the electrode in which a diameter of the electrode is expanded or reduced according to the rotation of the cam plate.

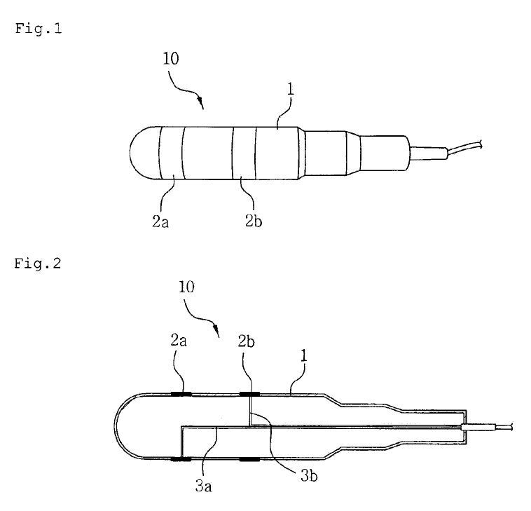

### DETAILED DESCRIPTION OF THE INVENTION

Hereinafter, a probe for an electric stimulator having a variable vaginal electrode according to a preferred embodiment of the present invention will be described with reference to accompanying drawings.

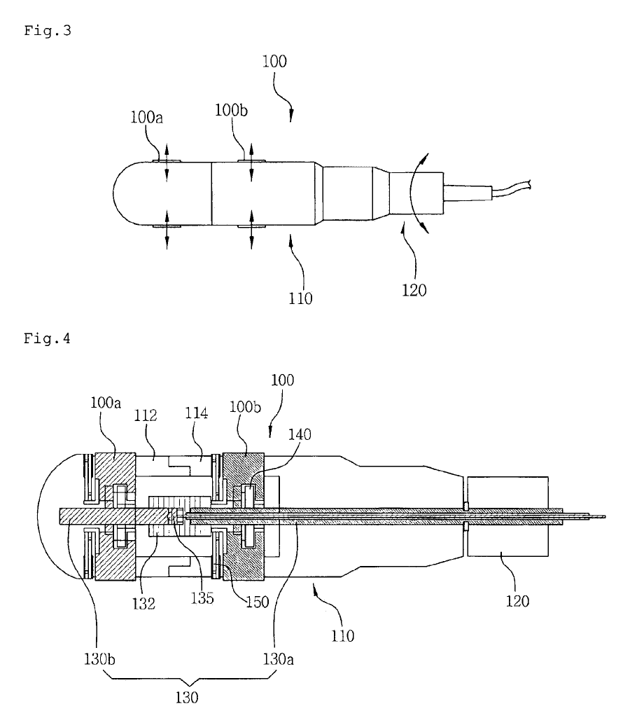

FIG. 3 is a schematic view showing an operation of a probe for an electric stimulator according to the present invention.

As shown in FIG. 3, differently from a conventional probe having a body integrated with a handle, a probe 100 for an electric stimulator according to the present invention has a structure, in which a rotatable handle 120 is separately provided below a body 110.

Accordingly, if the handle 120 is rotated to left or right, a diameter of electrodes 100a and 100b coupled to the body 110 is expanded or reduced.

Hereinafter, the inner structure of the probe will be described.

FIG. 4 is a view showing the inner structure of the probe for the electric stimulator according to the present invention.

As shown in FIG. 4, the probe for the electric stimulator according to the present invention includes the body 110 and the handle 120. The body 110 is divided into two parts, that is, a front part 112 and a rear part 114. The front part 112 is coupled to the rear part 114 through a conventional fitting scheme or a screw scheme. In addition, the handle 120 is rotatably coupled to the rear part 114.

Meanwhile, a shaft 130, which is fixed to the handle 120 and rotatable in the rotation direction of the handle 120, is accommodated in the probe 100. The shaft 130 includes a first shaft 130a, which extends from the handle 120 to a front end of the rear part 114, and a second shaft 130b, which extends from the front end of the rear part 114 to the inside of the front part 112.

The first shaft 130a is separated from the second shaft 130b, but the first and second shafts 130a and 130b are coupled to each other through a joint 132. Since the joint 132 includes insulating material, the joint 132 allows the first shaft 130a to be physically connected to the second shaft 130b without electrical connection.

A contact terminal 135 is provided between the first shaft 130a and the second shaft 130b. The contact terminal 135 is elastically supported at a front end of the first shaft 130a by a compression spring (not shown). A tip of the contact terminal 135 makes contact with the second shaft 130b, thereby achieving electrical connection. The contact terminal 135 makes contact with a power supply cable passing through the inside of the first shaft 130a such that electricity is applied to the second shaft 130b.

Cam plates 140 rotating together with the shaft 130 are coupled to an outer surface of the first shaft 130a and the second shaft 130b, respectively.

The cam plate 140 of the first shaft 130a is the same as the cam plate of the second shaft 130b, so the cam plate 140 of the first shaft 130a will be described as a representative example.

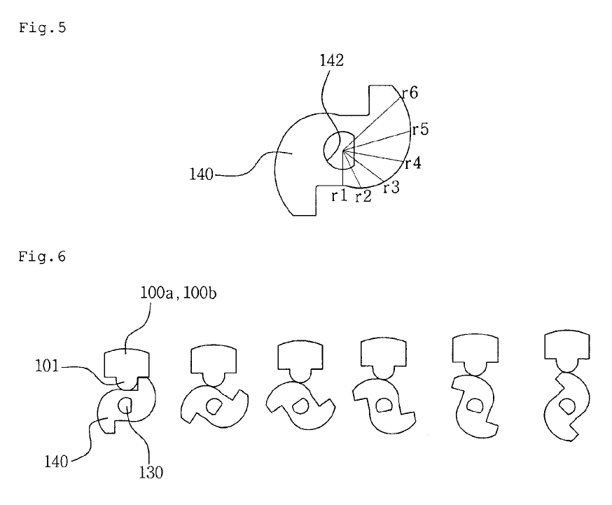

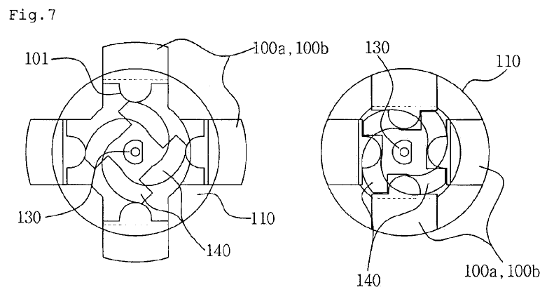

FIG. 5 is a view showing the cam plate shown in FIG. 4, FIG. 6 is a view showing an electrode which moves up and down according to a rotation of the cam plate, and FIG. 7 is a view showing the electrode in which a diameter of the electrode is expanded or reduced according to the rotation of the cam plate.

As shown in FIG. 5, the cam plate 140 has a substantially “S” shape, and a pair of cam plates 140 are coupled to the shaft 130 such that the cam plates 140 cross each other in the form of a pair.

That is, a through-hole 142 is formed in the center of the cam plate 140 such that the shaft 130 is inserted into the through-hole 142. A key hole, which is generally known in the art, is formed in the through-hole 142 such that the cam plate 140 rotates together with the shaft 130. In more detail, the cam plate 140 has symmetrical configuration such that the cam plate 140 has various radiuses r1, r2, r3, r4, r5 and r6, which are defined between the center of the through-hole 142 and the end of the cam plate 140, such that the radius of the cam plate 140 can be increased from one end to the other end of the cam plate 140.

The electrodes 100a and 100b making contact with the cam plate 140 are mounted at an outer circumference of the cam plate 140 such that the diameter of the electrode is expanded or reduced according to the rotation of the cam plate 140. That is, the electrodes 100a and 100b are mounted on opposite sides the cam plate 140 having the “S” shape, respectively. In addition, other electrodes 100a and 100b are mounted on opposite sides of other cam plate 140, respectively. A return spring 150 is installed between the electrodes 100a and 100b and the body 110 such that the electrodes 100a and 100b can elastically move. Preferably, a sliding protrusion 102 having a semicircular shape is integrally formed with a lower surface of the electrodes 100a and 100b such that the contact resistance between the electrodes 100a and 100b and the cam plate 140 is reduced and a plurality of electrodes 100a and 100b are densely disposed.

That is, according to the related art, the electrode has a cylindrical shape. However, according to the present invention, the electrode divided into a plurality of portions is disposed in a circular arrangement at equal intervals and the electrode moves up and down with respect to the body 110.

FIGS. 7 and 8 show the electrodes 100a and 100b, which move up and down according to the rotation of the cam plate 140. The diameter of the electrodes are expanded or reduced within a range of 30 mm to 40 mm. The above range is an illustrative purpose only, and the present invention is not limited thereto. According to another embodiment of the present invention, the diameter range of the electrode can be reduced or enlarged corresponding to the shape of the cam plate 140.

Hereinafter, the power supply line will be described.

In order to supply power, a power supply cable having a positive pole and a negative pole is prepared. In addition, one of the poles (for example, the negative pole) makes contact with the first shaft 130a, and the remaining one passes through the hollow formed in the first shaft 130a.

Accordingly, the negative pole connected to the first shaft 130a communicates with the cam plate 140 coupled to the first shaft 130a, and then communicates with the electrode 100b in the cam plate 140. The positive pole passing through the inside of the first shaft 130a is connected to the contact terminal 135, and the contact terminal 135 allows the positive pole to communicate with the second shaft 130b. Then, the second shaft 130b communicates with the electrode 100a through the cam plate 140. That is, the electrode 100b of the rear part 114 communicates with the negative pole, and the electrode 100a of the front part 112 communicates with the positive pole, thereby achieving the electrical stimulation treatment.

Although few embodiments of the disclosure have been shown and described, it would be appreciated by those skilled in the art that changes may be made in these embodiments without departing from the principles and sprit of the disclosure, the scope of which is defined in the claims and their equivalents.

What is claimed is:

1. A probe for an electric stimulator having a variable vaginal electrode, the probe comprising:
a body divided into a front part and a rear part; and
electrodes provided on the front and rear parts, respectively,
wherein a plurality of electrodes are provided in a circular arrangement such that the electrodes move up and down, a rotating handle is formed at a side of the body such that the handle rotates at the side of the body, a shaft coupled to the handle rotates when the handle rotates, so a cam plate coupled to the shaft rotates, and the rotated cam plate pushes the electrodes, thereby expanding or reducing a diameter of the electrodes.

2. The probe of claim 1, wherein a return spring is installed between the electrodes and the body.

3. The probe of claim 1, wherein the shaft includes a first shaft, which is fixed to the handle to extend toward a front end of the rear part, and a second shaft, which is connected to the first shaft through a joint to extend to an inside of the front part, and two cam plates having an S shape are coupled to the first shaft and the second shaft, respectively, while crossing each other such that a lower end of the electrodes is mounted on the two cam plates.

4. The probe of claim 3, wherein a power cable introduced through the handle has a first pole, which makes contact with the first shaft and communicates with the electrode of the rear part, and a second pole, which passes through an inside of the first shaft and makes contact with the second shaft through a contact terminal formed in the joint so that the second pole communicates with the electrode of the front part.

5. The probe of claim 3, wherein a sliding protrusion having a semicircular shape is formed at a lower surface of the electrodes

6. The probe of claim 4, wherein the joint includes insulating material, the contact terminal is elastically supported by a compression spring at a front end of the first shaft, and a tip of the contact terminal makes contact with the second shaft.
